(19) United States
(12) Patent Application Publication
(10) Pub. No.: US 2006/0134710 A1
(43) Pub. Date: Jun. 22, 2006

(54) WDR11 protein assay

(76) Inventor: Viorica Lopez-Avila, Sunnyvale, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/978,053

(22) Filed: Oct. 30, 2004

(51) Int. Cl. G01N 33/537; G01N 33/53; G01N 33/543

(52) U.S. Cl. 435/7.92

(57) ABSTRACT

An affinity-based analyte separation method of assaying a sample for a WDR11 protein. The method can be coupled to a variety of detection methods, including detection of an observable label or detection using mass spectrometry. The methods may be applied to assessing cancerous cells.

### FIELD OF THE INVENTION

The present invention generally relates to assays involving protein-protein interactions. More specifically, the invention relates to methods of determining the presence and/or quantity of a protein of interest in a sample.

### BACKGROUND

WD-repeat (WDR) proteins are a diverse superfamily of regulatory proteins that are found in a wide variety of organisms and that have several (e.g. 4-10) repeated WD motifs. Each WD motif (also called a Trp-Asp or WD-40 motif) typically is about 30 to about 40 amino acids long and has several conserved residues, e.g. the motif usually has conserved Trp-Asp residues at the C-terminal end of the motif. Investigations into the three dimensional structures of WDR proteins have found that repeated WD motifs form a series of four-stranded, antiparallel beta sheets folded into a higher-order structure termed a β-propeller (see. Smith et al., “The WD repeat: a common architecture for diverse functions,” Trends Biochem Sci 24:181-185(1999) ).

WDR proteins have been found to be involved in such functions as cell division, cytokinesis, cytoskeletal dynamics, apoptosis, chromatin modification, transcriptional mechanisms, nuclear export, RNA processing, protein trafficking, signal transduction, light signaling and vision, floral development, and meristem organization, as well as others. Inside the cell, the distribution of members of the WDR protein superfamily includes locations within the cytoplasm or nucleoplasm, linked to the cytoskeleton, or associated with membranes (e.g. via direct interactions with the membranes or via interactions with membrane proteins). To date, at least six human diseases have been linked to genes that code for putative proteins containing repeats of WD motifs. Further investigations of these proteins may provide clues into the mechanisms of these and potentially other human diseases.

Gliomas are central nervous system neoplasms derived from glial cells. Astrocytomas, glioblastoma multiforme, oligodendrogliomas, and ependymomas are all types of gliomas. Gliomas are known to occur in association with several well-defined hereditary tumor syndromes such as neurofibromatosis-1 and neurofibromatosis-2, tuberous sclerosis, Li-Fraumeni syndrome, and Turcot syndrome. Familial clustering of gliomas often occurs in the absence of these tumor syndromes, however. Large deletions at the INK4 tumor suppressor region involving the p16, p15, and p14 genes have been found in some families featuring both gliomas and skin melanomas.

Chernova et al. identified a reciprocal translocation in a glioblastoma cell line that involved regions on chromosomes 10 and 19. See Chernova et al.; “A novel member of the WD-repeat gene family, WDR11, maps to the 10q26 region and is disrupted by a chromosome translocation in human glioblastoma cells;” Oncogene 20: 5378-5392 (2001). By positional cloning of the translocation breakpoint, they identified a WD-repeat gene on chromosome 10, which they designated WDR11. The WDR11 gene contains 29 exons distributed over 58 kb and encodes a deduced 1,224-amino acid polypeptide with a calculated molecular mass of 137 kD. The polypeptide contains 6 putative WD40 repeats, a predicted transmembrane region, and a tyrosine kinase phosphorylation site. Northern blot analysis detected ubiquitous expression of 3 major transcripts (4.5, 2.7 and 2.0 kb) in all tissues tested. Because of the localization of WDR11 in a region frequently showing loss of heterozygosity in glioblastoma and because WDR11 is inactivated in glioblastoma cells, Chernova et al. considered WDR11 to be a candidate tumor suppressor gene involved in tumorigenesis of glial and other tumors. The WDR11 protein is thought to recognize and bind to some phosphorylated proteins and promote their ubiquitination and degradation. It may also participate in Wnt signaling.

Current methods for analyzing constituents of a complex sample find use in medical diagnostics and other fields, e.g. to measure the relative amounts of pre-determined analytes, e.g., proteins in blood and other bodily fluids. However, new and improved methodologies for complex sample analysis are desired.

References of interest include: published US patent applications 20010019829, 20010014461, 20020137106, 20020142343, 20020150927, 20020155509, 20020177242, 20020182649, 20020195555, 20030077616, 20030096224, 20030219731 and 20030027216; U.S. Pat. Nos. 6,630,358, 6,365,418 6,569,383 and 6,197,599; and Neubert et al, Anal. Chem. (2002) 74:3677-3683.

### SUMMARY OF THE INVENTION

The invention addresses the aforementioned deficiencies in the art, and provides for a novel affinity-based analyte separation method that can be coupled to a variety of detection methods, including detection of an observable label or detection using mass spectrometry.

In some embodiments, the invention provides an assay for detecting the presence of a WDR11 protein, a known tumor suppressor, in a sample. The assay generally includes contacting a SmB polypeptide with a sample and detecting any WDR11 protein bound to the SmB polypeptide to detect the presence of the WDR11 protein in the sample. In certain embodiments of the subject assays, the SmB polypeptide may be present on a solid support, e.g. directly or indirectly bound to the solid support, and the solid support is contacted with the sample. In certain other embodiments of the subject assays, the sample may be present on a solid support, and the SmB polypeptide may be labeled.

In certain embodiments, the method generally includes contacting a SmB polypeptide bound to a solid support with a sample to result in a WDR11/SmB complex. The WDR11/SmB complex is contacted with a digest reagent to result in WDR11 protein fragments, and then analyzing the WDR11 protein fragments, e.g. by mass spectrometry, to assess the WDR11 protein in the sample.

In particular embodiments, the method generally includes contacting a SmB polypeptide bound to a solid support with a sample to result in a WDR11/SmB complex. The WDR11/SmB complex is rinsed to remove sample not bound to the solid support, and then the WDR11/SmB complex is washed under conditions sufficient to release the WDR11 protein from the WDR11/SmB complex. The released WDR11 protein is then recovered in a wash-fraction. In some embodiments, the wash-fraction is contacted with a digest reagent to result in WDR11 protein fragments, and the WDR protein fragments are analyzed, e.g. by mass spectroscopy, to assess the WDR11 protein in the sample.

In another embodiment, the invention provides a method of assaying a population of glial cells, e.g. to determine if a glial cell is a cancerous glial cell. In general, this assay includes preparing a protein sample from the population of glial cells, contacting the protein sample with a SmB polypeptide; and detecting any WDR11 protein bound to the SmB polypeptide. In particular embodiments, results obtained from the method are used to determine whether the population of glial cells includes cancerous glial cells. The method may further include comparing results obtained from the assay to control results. Control results may be obtained by performing the assay using a sample known to contain the WDR11 protein, and/or a sample known to contain no WDR11 protein. A reduced level of WDR11 protein bound to the SmB polypeptide, compared to the level of WDR11 protein in a normal control, generally indicates that the population of glial cells includes cancerous glial cells. In various embodiments, the protein sample may be prepared from a population of cultured cells, or, in alterative embodiments, a sample of brain tissue.

Additional novel features of this invention shall be set forth in part in the descriptions and examples that follow and in part will become apparent to those skilled in the art upon examination of the following specifications or may be learned by the practice of the invention. The invention may be realized and practiced by means of the instruments, combinations, compositions and methods particularly pointed out in the appended claims.

### BRIEF DESCRIPTION OF THE DRAWINGS

These and other features of the invention will be understood from the description of representative embodiments of the method herein and the disclosure of illustrative apparatus for carrying out the method, taken together with the Figures, wherein

FIG. 1 illustrates a method of assaying a sample for WDR11 protein in accordance with the present invention.

FIG. 2 depicts a method of assaying a population of glial cells in accordance with the present invention.

To facilitate understanding, identical reference numerals have been used, where practical, to designate corresponding elements that are common to the Figures. Figure components are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible. However, the sequence described below is preferred.

It must be noted that, as used in the specification and the appended claims, the singular forms “a,” “an” and “the” include plural referents unless the context clearly dictates otherwise. Thus, for example, reference to “a solid support” includes a plurality of solid supports. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

In this specification and in the claims that follow, reference will be made to a number of terms that shall be defined to have the following meanings unless a contrary intention is apparent.

The term “sample” as used herein relates to a material or mixture of materials, typically, although not necessarily, in fluid form, e.g., aqueous, containing one or more components of interest. Samples may be derived from a variety of sources such as from food stuffs, environmental materials, a biological sample such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components).

“Bound” may be used herein to indicate direct or indirect attachment. “Bound” (or “bonded”) may refer to the existence of a chemical bond directly joining two moieties or indirectly joining two moieties (e.g. via a linking group or other entity). The chemical bond may be a covalent bond, an ionic bond, a coordination complex, hydrogen bonding, van der Waals interactions, hydrophobic stacking, or non-covalent bond, or may exhibit characteristics of multiple types of chemical bonds. In certain instances, “bound” includes embodiments where the attachment is direct and also embodiments where the attachment is indirect. Depending on the context, “connected”, “linked”, or other like term indicates that two groups are bound to each other, wherein the attachment may be direct or indirect, wherein the attachment may be reversible or irreversible. “Immobilized” references a group that is bound to another moiety, e.g. covalently, non-covalently, reversibly, non-reversibly. Reversible binding indicates binding that typically may be released, e.g. by changing the conditions to promote release of a bound species. An example of reversible binding is binding of an analyte to a capture agent followed by elution of the analyte by changing the conditions under which the contact is occurring, e.g. changing pH, temperature, ionic strength, salt concentration Irreversible binding indicates that the bound species remains bound during normal performance of the methods of the present invention.

The term “analyte” is used herein to refer to a known or unknown component of a sample. The analyte may be specifically bound to another component in solution or on a support, e.g. to a capture agent on a substrate surface. In particular embodiments, the analyte (or a portion of the analyte) and the capture agent are members of a specific binding partner pair. In general, analytes are biopolymers, i.e., an oligomer or polymer such as an oligonucleotide, a peptide, a polypeptide, an antibody, or the like. In this case, an “analyte” is referenced as a moiety in a mobile phase (e.g., fluid), to be detected by a “capture agent” which, in some embodiments, is bound to a substrate, or in other embodiments, is in solution. However, either of the “analyte” or “capture agent” may be the one which is to be evaluated by the other (thus, either one could be an unknown mixture of analytes, e.g., polypeptides, to be evaluated by binding with the other). In particular embodiments, the capture agent may be labeled with a label which has an observable characteristic, providing for detection of the analyte or capture agent by detecting the detectable characteristic.

The term “capture agent” refers to an agent that binds an analyte through an interaction that is sufficient to permit the agent to bind and concentrate the analyte from a homogeneous mixture of different analytes. The binding interaction may be mediated by an affinity region of the capture agent. Representative capture agents include polypeptides and polynucleotides, for example antibodies, peptides or fragments of double stranded DNA may employed. Accordingly, the term “capture agent” refers to a molecule or a multi-molecular complex which can specifically bind an analyte, e.g. specifically bind an analyte for the capture agent with a dissociation constant (K_{D}) of less than about 10⁻⁶ M (e.g. 10⁻⁷ M) without binding to other targets.

Capture agents usually “specifically bind” one or more analytes. The term “specific binding” refers to the ability of a capture agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the affinity between a capture agent and analyte when they are specifically bound in a capture agent/analyte complex is characterized by a K_{D} (dissociation constant) of less than about 10⁻⁶ M (e.g. less than about 10⁻⁷ M, less than about 10⁻⁸ M, less than about 10⁻⁹ M) and typically greater than about 10⁻¹⁰ M.

The term “capture agent/analyte complex” is a complex that results from the specific binding of a capture agent with an analyte, i.e., a “binding partner pair”. A capture agent and an analyte for the capture agent specifically bind to each other under “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens, are well known in the art (see, e.g., Harlow and Lane (Antibodies: A Laboratory Manual Cold Spring Harbor Laboratory, Cold Spring Harbor, N.Y. (1989)). Conditions suitable for specific binding typically permit capture agents and target pairs that have a dissociation constant (K_{D}) of less than about 10⁻⁶ M to bind to each other, but not with other capture agents or targets.

As used herein, “binding partner pairs” and equivalents refer to pairs of molecules that can be found in a capture agent/analyte complex, i.e. exhibit specific binding with each other.

The phrase “surface-bound capture agent” refers to a capture agent that is immobilized on a surface of a solid support, where the support can have a variety of configurations, e.g., a sheet, bead, or other structure, such as a plate with wells. In certain embodiments, the collections of capture agents employed herein are present on a surface of the same support, e.g., in the form of an array. In particular embodiments of the invention, the capture agent (e.g. SmB polypeptide, or an antibody) may be bound to a surface, e.g. of a solid support. In such embodiments, the capture agent may be bound directly to the surface or indirectly, e.g. via a linker or a binding partner. For example, in an embodiment in which an anti-WDR11 antibody is bound to the surface (directly or indirectly), a WDR11 protein may then bind to the anti-WDR11 antibody and thus become indirectly bound to the surface via the anti-WDR11 antibody.

The term “pre-determined” refers to an element whose identity is known prior to its use. For example, a “pre-determined analyte” is an analyte whose identity is known prior to any binding to a capture agent. An element may be known by name, sequence, molecular weight, its function, or any other attribute or identifier. In some embodiments, the term “analyte of interest”, i.e. a known analyte that is of interest, is used synonymously with the term “pre-determined analyte”.

The term “mixture”, as used herein, refers to a combination of elements, e.g., capture agents or analytes, that are interspersed and not in any particular order. A mixture is homogeneous and not spatially separable into its different constituents. Examples of mixtures of elements include a number of different elements that are dissolved in the same aqueous solution, or a number of different elements attached to a solid support at random or in no particular order in which the different elements are not specially distinct. In other words, a mixture is not addressable. To be specific, an array of capture agents, as is commonly known in the art and described below, is not a mixture of capture agents because the species of capture agents are spatially distinct and the array is addressable.

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally. Put in other terms, “purified” or “isolated” references a substance from which a substantial portion of the sample in which the substance resides has been separated from the substance, leaving a greater proportion of the purified substance than prior to the separation.

The term “assessing” includes any form of measurement, and includes determining if an element is present or not. The terms “determining”, “measuring”, “evaluating”, “assessing” and “assaying” are used interchangeably and may include quantitative and/or qualitative determinations. Assessing may be relative or absolute. “Assessing the presence of” includes determining the amount of something present, and/or determining whether it is present or absent.

The terms “antibody” and “immunoglobulin” are used interchangeably herein to refer to a capture agent that has at least an epitope binding domain of an antibody. These terms are well understood by those in the field, and refer to a protein containing one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG1, IgG2, IgG3, IgG4), delta, epsilon and mu heavy chains or equivalents in other species. Full-length immunoglobulin “light chains” (of about 25 kDa or about 214 amino acids) comprise a variable region of about 110 amino acids at the NH2-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding partner pairs, e.g., biotin (member of biotin-avidin specific binding partner pair), and the like. The antibodies may also be bound to a solid support, including, but not limited to, polystyrene plates or beads, and the like. Also encompassed by the terms are Fab′, Fv, F(ab′)2, and or other antibody fragments that retain specific binding to antigen.

Antibodies may exist in a variety of other forms including, for example, Fv, Fab, and (Fab′)2, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)). Monoclonal antibodies and “phage display” antibodies are well known in the art and encompassed by the term “antibodies”.

A “biopolymer” is a polymer of one or more types of repeating units, regardless of the source. Biopolymers may be found in biological systems and particularly include polypeptides and polynucleotides, as well as such compounds containing amino acids, nucleotides, or analogs thereof. The term “polynucleotide” refers to a polymer of nucleotides, or analogs thereof, of any length, including oligonucleotides that range from 10-100 nucleotides in length and polynucleotides of greater than 100 nucleotides in length. The term “polypeptide” refers to a polymer of amino acids of any length, and encompasses “peptide,” which references a polymer of amino acids in the range from 6-50 amino acids in length. In general, polypeptides may be of any length, e.g., greater than 2 amino acids, greater than 4 amino acids, greater than about 10 amino acids, greater than about 20 amino acids, greater than about 50 amino acids, greater than about 100 amino acids, greater than about 300 amino acids, usually up to about 500 or 1000 or more amino acids. “Peptides” are generally greater than 2 amino acids, greater than 4 amino acids, greater than about 10 amino acids, greater than about 20 amino acids, usually up to about 50 amino acids. In some embodiments, peptides are between 5 and 30 amino acids in length.

The terms “polypeptide” and “protein” are used interchangeably. The term “polypeptide” includes polypeptides in which the conventional backbone has been replaced with non-naturally occurring or synthetic backbones, and peptides in which one or more of the conventional amino acids have been replaced with one or more non-naturally occurring or synthetic amino acids. The term “fusion protein” or grammatical equivalents thereof references a protein composed of a plurality of polypeptide components, that while not attached in their native state, are joined by their respective amino and carboxyl termini through a peptide linkage to form a single continuous polypeptide. Fusion proteins may be a combination of two, three or even four or more different proteins. The term polypeptide includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, and the like.

As known in the art “similarity” between two polypeptides is determined by comparing the amino acid sequence and its conserved amino acid substitutes of one polypeptide to the sequence of a second polypeptide. Such conservative substitutions include those described by Dayhoff and by Argos (see Dayhoff, The Atlas of Protein Sequence and Structure 5 (1978); Argos, EMBO J. 8: 779-785 (1989) ). For example, amino acids belonging to one of the following groups represent conservative changes:
ala, pro, gly, gln, asn, ser, thr;
cys, ser, tyr, thr;
val, ile, leu, met, ala, phe;
lys, arg, his;
phe, tyr, trp, his; and
asp, glu.

(Note that these grouping are examples; other groupings may represent more relevant choices.)

“Similarity” or “identity” refers to sequence conservation, or “homology”, between two or more peptides or two or more nucleic acid molecules, normally expressed in terms of percentages. When a position in the compared sequences is occupied by the same base or amino acid (“residue”), then the molecules are identical at that position. When a position in two compared peptide sequences is occupied by an amino acid with similar physical properties (a conservative substitution as determined by a given scoring matrix; similarity is thus dependent on the scoring matrix chosen), then the molecules are similar at that position. The percent identity or similarity can be maximized by aligning the compared sequences alongside each other, sliding them back and forth, and conservatively introducing gaps in the sequences where necessary. The percent identity is calculated by counting the number of identical aligning residues dividing by the total length of the aligned region, including gaps in both sequences, and multiplying by 100. Identity would thus be expressed as, e.g., “60% identity over 200 amino acids,” or “57% identity over 250 amino acids.” Similarity is calculated by counting both identities and similarities in the above calculation. For example, the alignment below has 37.5% sequence identity over 56 amino acids ((21 identities/56 residues)×100%), where 56 is the total length of the aligned region.

| | |
|---|---|
| RTPSDKPVAH--VANPQLQWLNRRANALLANGVE-RDNQLVV--EGLYLIYSQVLF | 56 resid. |
| \| \| \| \| \|\| \| \| \| \| \|\| \| \|\| \|\|\| \| \| \| | 21 ident. |
| RAPFKKSWAYLQVAKHKLSW-NK--DGIL-HGVRYQDGNLVIQFPGLYFIICQLQF | 56 resid. |

As a further example, the same alignment below has 55.4% sequence similarity over 56 amino acids ((31 similarities/56 residues)×100%), where 56 is the total length of the aligned region. In this example, conservative substitutions are indicated by a plus sign and the total similarities is given by the sum of the identities and the conservative substitutions. (As noted above, determination of conservative substitutions is dependent on the scoring matrix chosen. The same alignment below may yield a different value for percent similarity using a different scoring matrix.)

| | |
|---|---|
| RTPSDKPVAH--VANPQLQWLNRRANALLANGVE-RDNQLVVE--GLYLIYSQVLF | 56 resid. |
| R P K A+ VA +L W N N+ +L +GV +D LV++ GLY I Q+ F | 31 simil. |
| RAPFKKSWAYLQVAKHKLSW-NK--DGIL-HGVRYQDGNLVIQFPGLYFIICQLQF | 56 resid. |

Both of the sequences in the aligned region may be contained within longer, less homologous sequences. “Unrelated” or “non-homologous” sequences typically share less than 40% identity at the peptide level, preferably less than 25% identity.

The term “array” encompasses the term “microarray” and refers to an ordered array of capture agents for binding to aqueous analytes and the like. An “array,” includes any two-dimensional or substantially two-dimensional (as well as a three-dimensional) arrangement of spatially addressable regions (i.e., “features”) containing capture agents, particularly antibodies, and the like. Where the arrays are arrays of proteinaceous capture agents, the capture agents may be adsorbed, physisorbed, chemisorbed, or covalently attached to the arrays at any point or points along the amino acid chain. In some embodiments, the capture agents are not bound to the array, but are present in a solution that is deposited into or on features of the array.

Any given substrate may carry one, two, four or more arrays disposed on a surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of the same or different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained capture agent.

An array is “addressable” when it has multiple regions of different moieties (e.g., different capture agent) such that a region (i.e., a “feature” or “spot” of the array) at a particular predetermined location (i.e., an “address”) on the array will detect a particular sequence. Array features are typically, but need not be, separated by intervening spaces.

The subject array may be an array of features, each feature corresponding to a “fluid-retaining structure”, e.g., a well, wall, hydrophobic barrier, or the like. Such arrays are well known in the art, and include 24-well, 48-well, 96-well, 192-well, 384-well and 1536-well microtiter plates, or multiple thereof. In certain embodiments, the features are delineated by a hydrophobic chemical boundary, and, accordingly, the array substrate may be planar and contain features containing a hydrophobic boundary. Features may be delineated by drawing lines between them with a hydrophobic pen (e.g., a PAP PEN from Newcomer Supply, Middleton, Wis.), for example. Other fluid retaining structures are well known in the art and include physical and chemical barriers. On one embodiment, the fluid retaining structure is formed by a bead of hydrophobic material, e.g., a bead of a viscose silicone material, around a fluid-retaining area. Capture agents may be present in the fluid retaining structure, but not necessarily bound to the surface of the array substrate.

An “array layout” refers to one or more characteristics of the features, such as feature positioning on the substrate, one or more feature dimensions, and an indication of a moiety at a given location.

The term “MALDI sample plate” refers to a device that is removably insertable into a MALDI ion source for a mass spectrometer and contains a substrate having a surface for presenting analytes for ionization by a laser. As will be described in greater below, a MALDI sample plate may contain a plurality of features, i.e., discrete, addressable regions, each containing a different analyte for ionization by the laser of the MALDI mass spectrometer.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

Methods of Detecting the WDR11 Protein in a Sample

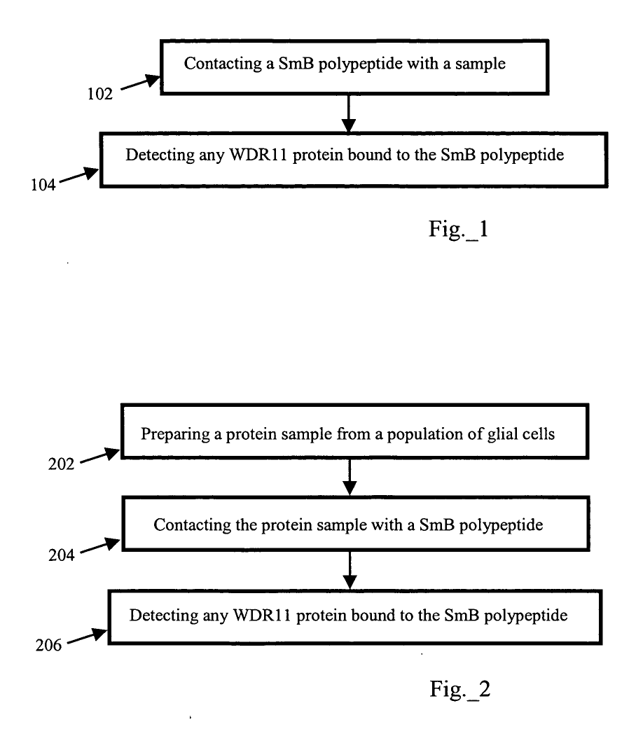

As described below, a previously unknown interaction between an SmB polypeptide, a core protein of the snRNP complex, and a WDR11 protein, a tumor suppressor thought to be involved in tumorigenesis of glial tumors (Chemova et al, Oncogene 2001, 20:5378-5392), is employed in analyzing a sample in order to detect the presence of a WDR11 protein in the sample. Accordingly, as shown in FIG. 1, the instant invention provides a method of assaying a sample for a WDR 11 protein including contacting a SmB polypeptide with the sample 102 and detecting any WDR11 protein bound to the SmB polypeptide 104. In typical embodiments, the SmB polypeptide is contacted with a WDR11 protein in the sample under conditions sufficient to allow for binding of the WDR11 protein in the sample to the SmB polypeptide. These methods find use in assays for detecting cancerous glial cells and assays for diagnosing a glial tumor in a subject, which assays will be described in greater detail below.

As noted above, a WDR11 protein is a tumor suppressor protein implicated in tumorigenesis of glial tumors and other tumors associated with alterations in the distal region of chromosome 10q (e.g., at approximately 10g25-26; Chemova et al., supra; Katoh et al, Int. J. Oncol. 2003 22:1155-9, and others). An exemplary amino acid sequence and encoding cDNA sequence for a WDR11 protein is set forth in NCBI's GenBank database accession number BC071564 (GID 48734941), which database entry (including any polynucleotide and polypeptide sequences or annotation set forth therein) is incorporated by reference herein in its entirety. The function of WDR11 protein is summarized in NCBI's OMIM database accession number 606417, which database entry is incorporated by reference herein in its entirety. As would be recognized by one of skill in the art, because the sequence of a protein may vary from individual to individual (e.g., through polymorphisms in the gene encoding the protein, or by modification of the amino acid sequence of the protein by use of genetic engineering techniques), the WDR11 protein present in a sample may have an amino acid sequence that is identical or different to that set forth in Genbank database access number BC071564, for example, more than about 95% identical, more than about 96% identical, more than about 97% identical, more than about 98% identical, or more than about 99% identical, said percent identity being determined over at least about 60%, e.g. at least about 70%, at least about 80%, at least about 90%, at least about 95%, at least about 98% of the total protein sequence set forth in Genbank database access number BC071564.

The SmB polypeptide is a well known, previously described core component of the mammalian spliceosome (see, e.g., van Dam et al, EMBO J. 1989 8:3853-3860). A SmB polypeptide employed in the subject methods generally contains at least a C-terminal region of (e.g., the C-terminal 10, 20, 25 or 30 amino acids, including all the contiguous amino acids of) a full length mammalian, e.g., human, SmB or SmB′ polypeptide (where Smb and SmB′ polypeptides are splice variants encoded by the same gene). Exemplary amino acid sequences for full length SmB polypeptides are found in NCBI's GenBank database entries defined by accession numbers A35448 (GID 112305; rat); AAH03530 (GID 45708426; human); CAG33250 (GID 48146055; wallaby), NP_{—}033251 (GID 6678053; mouse); Q9TU66 (GID 10720265; opossum), for example. These database entries (including any polynucleotide and polypeptide sequences or annotation set forth therein) are hereby incorporated by reference in their entireties. A SmB polypeptide for use in the subject methods may be designed in view of any these sequences. In certain embodiments, a SmB polypeptide may be a fusion polypeptide, as described above.

In general, methods for producing and isolating polypeptides for employment in the subject methods are well known in the art and described in, for example, Ausubel, et al, (Short Protocols in Molecular Biology, 5th ed., Wiley & Sons, 2002) and Sambrook, et al. (Molecular Cloning: A Laboratory Manual, Third Edition, (2001) Cold Spring Harbor, N.Y.) and need not be discussed in any more detail than that set forth above. In certain embodiments a SmB polypeptide may be employed in the subject methods. In these methods, the SmB polypeptide may be chemically synthesized by routine methods by a peptide synthesizer.

Depending on the method used, the SmB polypeptide may be linked (covalently or non-covalently, directly or indirectly) to a solid support. A “solid support” or “carrier” is intended to encompass any support capable of binding a SmB polypeptide. Well-known supports or carriers, include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention. The support material can have virtually any possible structural configuration as long as the coupled molecule is capable of binding to a WDR11 protein. Thus, the support configuration can be spherical, as in a bead, or cylindrical, as in the inside surface of a test tube, or the external surface of a rod. Alternatively, the surface can be flat, such as a sheet, culture dish, test strip, etc. Those skilled in the art will know many other suitable carriers for binding antibody, peptide or antigen, or can ascertain the same by routine experimentation.

The subject assays for detecting the presence of a WDR11 protein in a sample may be done using one or more of a number of different test formats. The specific examples set forth below are intended to exemplify the invention and should no limit the invention in any way. For example, methods in accordance with the present invention may include (but are not limited to) assaying a sample employing such methods as competitive and non-competitive assay systems such as western blotting systems, radioassay systems, ELISA (enzyme linked immunosorbent assay) systems, “sandwich” assay systems, diffusion assay systems, affinity chromatography systems, and the like. Such assay systems are routine and well known in the antibody detection arts and can be readily adapted for the subject methods (see, e.g., Ausubel et al, eds, 1994, Current Protocols in Molecular Biology, Vol. 1, John Wiley & Sons, Inc., New York). In exemplary embodiments a subject assay may formatted as an ELISA assay, a sandwich assay, or a dipstick assay (similar to that used for pregnancy tests). As would be recognized by one of skill in the art, in many of the subject methods, either the SmB polypeptide or the sample may be bound to a solid support prior to initiating the subject methods.

An ELISA assay involves contacting a sample (e.g., a lysed cell extract or the like) bound to a solid support (generally a well of a multi-well plate) with a SmB polypeptide conjugated to a detectable compound such as an enzyme (e.g., horseradish peroxidase or alkaline phosphatase), and, after washing, detecting the presence of any SmB polypeptide bound to the solid support (via any WDR11 protein in the sample) by contacting the solid support with a substrate for the enzyme to produce a detectable signal.

A sandwich assay involves contacting a sample with a SmB polypeptide that is bound to a solid support, and, after washing, detecting the presence of any WDR11 protein bound to the SmB polypeptide using a detectably labeled anti-WDR11 antibody. In an alternative embodiment, the anti-WDR11 antibody is the moiety that is bound to the solid support, and the SmB polypeptide is detectably labeled; such that the sandwich assay involves contacting a sample with the anti-WDR11 antibody that is bound to a solid support, and, after washing, detecting the presence of any WDR11 protein bound to the anti-WDR11 antibody using the detectably labeled SmB polypeptide.

In a further exemplary embodiment, a liquid sample is applied to one end of a test strip containing a test region containing a SmB polypeptide bound thereto, and the sample (including any WDR11 protein in the sample) is allowed to migrate along the test strip by capillary action or lateral flow. Any WDR11 protein is “captured” in the test region, and detected using a detectably labeled anti-WDR11 antibody for example. Methods and devices for lateral flow separation, detection, and quantitation are known in the art. See, e.g., U.S. Pat. Nos. 5,569,608; 6,297,020; and 6,403,383.

In certain embodiments, results obtained from the assay are compared to results obtained from a control assay. In particular embodiments, the control assays may be run in parallel or in series with and may be otherwise identical to the assays described above, except the control assays may employ a sample that is known to contain WDR11 protein or a sample that is known to not contain any WDR11 protein.

In some embodiments, the invention provides an assay for detecting the presence of a WDR11 protein, a known tumor suppressor, in a sample. The assay generally includes contacting a SmB polypeptide with a sample and detecting any WDR11 protein bound to the SmB polypeptide to detect the presence of the WDR11 protein in the sample. The contacting may be done under conditions suitable for binding of the WDR11 protein in the sample to the SmB polypeptide. In certain embodiments, the assay provides results which may be compared to results obtained from a control assay, for example, an assay in which the sample is known to contain or is known not to contain any WDR11 protein. The assay may be in a variety of formats, such as a sandwich binding assay or an enzyme-linked immunoabsorbance assay (ELISA), for example, and may employ an anti-WDR11 antibody, e.g., a labeled anti-WDR11 antibody. In certain embodiments of the subject assays, the SmB polypeptide may be present on a solid support, and the solid support is contacted with the sample. In certain other embodiments of the subject assays, the sample may be present on a solid support, and the SmB polypeptide may be detectably labeled.

In particular embodiments, one or more of the components (e.g. the SmB polypeptide, analytes in the sample, an anti-WDR11 antibody) may be labeled. In such embodiments, the component has an attached label which has an observable characteristic, e.g. a chromophore, a fluorophore, a radioisotope, an enzyme, or a mass label, or other label having an observable characteristic. Detection of the labeled component is then accomplished by monitoring the observable characteristic using any effective methods and/or apparatus for monitoring, such as is well known in the art.

In some embodiments, the assay generally includes contacting a SmB polypeptide bound to a solid support with a sample and detecting any WDR11 protein bound to the SmB polypeptide to assess the sample for the WDR11 protein. In such embodiments, contacting the SmB polypeptide with the WDR11 protein present in the sample results in a WDR11/SmB complex bound to the solid support. The WDR11/SmB complex includes the WDR11 protein bound to the SmB polypeptide. In certain embodiments of the invention, the WDR11/SmB complex is contacted with a digest reagent. The SmB polypeptide may also be cut in some embodiments to yield SMB fragments. The digest reagent may be any agent that cuts the WDR11 protein to result in WDR11 protein fragments. Agents capable of cutting proteins are known in the art and include chemical cleavage reagents and enzymatic cleavage reagents, such as trypsin, chymotrypsin, pepsin, elastase, or any other endopeptidase, in particular, an endopeptidase which cuts at a specific site. For example, trypsin will typically cut a protein at an accessible arginine or lysine residue. In such methods according to the present invention, detecting the WDR11 protein includes contacting the WDR11/SmB complex with a digest reagent to result in WDR11 protein fragments and then analyzing the WDR11 protein fragments. In particular such methods, the WDR11 protein fragments are analyzed using mass spectrometry, e.g. employing methods such as MALDI-MS (matrix assisted desorption/ionization mass spectrometry) or ESI-MS (electrospray ionization mass spectrometry).

In other embodiments, the method of assaying a sample includes contacting a SmB polypeptide bound to a solid support with the sample to result in a WDR11/SmB complex bound to the solid support, and then detecting any WDR11 protein bound to the SmB polypeptide. The WDR11/SmB complex is typically rinsed to remove sample not bound to the solid support. The WDR11/SmB complex is washed (e.g. contacted with a buffer) under conditions sufficient to release the WDR11 protein from the WDR11/SmB complex, and then a wash-fraction is recovered which includes the WDR11 protein. In such embodiments, the wash-fraction is contacted with a digest reagent to result in WDR11 protein fragments; and the WDR11 protein fragments are then analyzed. In particular such methods, the WDR11 protein fragments are analyzed using mass spectrometry, e.g. employing methods such as MALDI-MS (matrix assisted desorption/ionization mass spectrometry) or ESI-MS (electrospray ionization mass spectrometry).

Assays for Detecting a Cancerous Glial Cell

As described above, the invention provides a method for detecting WDR11 protein in a sample. Since WDR11 protein is a tumor suppressor whose expression is inversely correlated with (i.e., the WDR11 protein is reduced or not expressed in) glial cancer, the invention also provides an assay for detecting a cancerous glial cell. This assay method, illustrated in FIG. 2, generally involves obtaining a sample from glial cells 202, wherein obtaining the sample encompasses any method of sample preparation resulting in proteins from the glial cells becoming available for binding to SmB in the assay, e.g. lysing the cells to release WDR11 protein, recovering a fraction from a homogenized sample of cells wherein the fraction includes WDR11 protein, etc. The sample used in the assay may be a sample obtained from cultured cells, or, in alterative embodiments, a sample obtained from brain tissue. In typical embodiments, the method includes contacting the sample (e.g., a cell extract or the like) with a SmB polypeptide 204 under conditions sufficient to allow binding of any WDR11 protein in the sample to the SmB polypeptide, and detecting any WDR11 protein bound to the SmB polypeptide 206. In certain embodiments, the absence or reduced concentration of WDR11 protein bound to the SmB polypeptide, compared to the level of WDR11 protein in a normal control, is an indicator that the cells from which the sample was obtained are cancerous.

Again, in certain embodiments, results obtained from this assay are compared to results obtained from a control assay. In particular embodiments, the control assays may be run in parallel with and may be otherwise identical to the assays described above, except the control assays may employ a sample of a cell that is known to contain WDR11 protein or a sample that is known to not contain any WDR11 protein.

In some embodiments, the assay method generally involves obtaining a sample (e.g., a cell extract or the like) from glial cells, contacting the sample with a SmB polypeptide bound to a solid support under conditions sufficient to allow binding of any WDR11 protein in the sample to the SmB polypeptide to result in a WDR11/SmB complex bound to the solid support, and then detecting the WDR11 protein.

In particular embodiments, one or more of the components (e.g. the SmB polypeptide, analytes in the sample, an anti-WDR11 antibody) may be labeled. In such embodiments, the component has an attached label which has an observable characteristic, e.g. a chromophore, a fluorophore, a radioisotope, an enzyme, or a mass label, or other label having an observable characteristic. Detection of the labeled component is then accomplished by monitoring the observable characteristic using any effective methods and/or apparatus for monitoring, such as is well known in the art.

In alternative embodiments, the subject methods find use in diagnosing cancer (e.g., glial cancer) in a subject. These methods generally involve contacting a SmB polypeptide with a sample of the subject (typically a human subject) under conditions sufficient to allow binding of the WDR11 protein in the sample to the SmB polypeptide, and detecting any WDR11 protein bound to the SmB polypeptide. Again, the absence (or a reduced level) of WDR11 protein bound to the SmB polypeptide indicates a diagnosis of cancer (e.g., glial cancer). In these embodiments the sample tested may be a sample of brain tissue, for example, or a sample of any other tissue suspected of containing cancerous cells.

In certain embodiments, the method may employ an array having a plurality of array features on an array substrate. At least one of the array features comprises SmB polypeptide bound to the array substrate. The array further comprises a plurality of additional capture agents, each of the plurality of array features comprising a capture agent bound to the array substrate. In such embodiments, a method of analyzing a sample comprises contacting the array with the sample and detecting one or more analytes from the sample bound to one or more of the plurality of array features. In certain embodiments, the array substrate is a multi-well plate, and each array feature is disposed in its own well of the multi-well plate. In some embodiments, detecting one or more analytes comprises analyzing the analytes using mass spectrometry. In some embodiments, the detection provides a result that may used, e.g. in assessing WDR11 protein in the sample.

The subject methods may be employed in a variety of diagnostic, drug discovery, and research applications that include, but are not limited to, diagnosis or monitoring of a disease or condition (where analytes that are markers for the disease or condition are assessed), discovery of drug targets (an analyte whose level is modulated in a disease or condition is a drug target), drug screening (where the effects of a drug are monitored by assessing the levels of analytes), protein fingerprinting (where the profile, i.e., the expression levels of analytes are assessed in a variety of diseases or artificial conditions and the profile provides a fingerprint for that disease or condition), determining drug susceptibility (where drug susceptibility is associated with a particular profile of analytes), discovery of new binding partners (where an analyte that binds to a capture agent has not been previously identified) and research (where is it desirable to know the relative concentrations of a number of analytes in a sample, or, conversely, the relative levels of an analyte in two or more samples).

The invention further provides a kit for testing for the presence of WDR11 protein in a sample, the kit comprising: a SmB polypeptide. The kit may also contain an anti-WDR11 antibody and/or an anti-SmB antibody. The SmB polypeptide, anti-WDR11 antibody or anti-SniB antibody, if present, may be attached to a solid support. The kit may contain instructions for detecting the presence of a WDR11 protein in a sample. The kit may further contain reagents for sample preparation, and/or buffers for washing the solid support.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties.

What is claimed is:

1. A method of assaying a sample for WDR11 protein, the method comprising
contacting a SmB polypeptide with the sample, and
detecting any WDR11 protein bound to the SmB polypeptide.

2. The method of claim 1, wherein the contacting is performed under conditions sufficient to allow binding of any WDR11 protein in the sample to the SmB polypeptide.

3. The method of claim 1, wherein the detecting provides a result, the method further comprising comparing the result with a control result to assess the WDR11 protein in the sample.

4. The method of claim 1, wherein the method is selected from an ELISA method, a sandwich binding assay, a western blot assay, a radioassay, a diffusion assay, or an affinity chromatography method.

5. The method of claim 1, wherein the SmB polypeptide is immobilized on a solid support, and the solid support is contacted with the sample.

6. The method of claim 1, wherein the SmB polypeptide is labeled with a label having an observable characteristic, and detecting any WDR11 protein comprises detecting the observable characteristic.

7. The method of claim 6, wherein the label is selected from the group consisting of a fluorophore, a chromophore, a radioisotope, an enzyme, and a mass label.

8. The method of claim 1, wherein contacting the SmB polypeptide with the sample comprises immobilizing WDR11 protein onto a solid support and then contacting the solid support with a solution comprising the SmB polypeptide.

9. The method of claim 1, wherein the SmB polypeptide is bound to a solid support and contacting results in a WDR11/SmB complex bound to the solid support.

10. The method of claim 9, wherein detecting comprises contacting the WDR11/SmB complex with a digest reagent to result in WDR11 protein fragments and then analyzing the WDR11 protein fragments.

11. The method of claim 10 wherein analyzing the WDR11 protein fragments is accomplished using mass spectrometry.

12. The method of claim 9, wherein detecting comprises:
rinsing the WDR11/SmB complex to remove sample not bound to the solid support,
washing the WDR11/SmB complex under conditions sufficient to release the WDR11 protein from the WDR11/SmB complex and recovering a wash-fraction including the WDR11 protein,
contacting the wash-fraction with a digest reagent to result in WDR11 protein fragments; and
analyzing the WDR11 protein fragments.

13. The method of claim 12 wherein analyzing the WDR11 protein fragments is accomplished using mass spectrometry.

14. A method of assaying a population of glial cells, the method comprising:
preparing a protein sample from said population;
contacting the protein sample with a SmB polypeptide; and
detecting any WDR11 protein bound to the SmB polypeptide,

15. The method of claim 14, wherein the method is used to diagnose a glial cell cancer.

16. The method of claim 14, wherein detecting provides a result, the method further comprising comparing the result with a control result to assess the WDR11 protein in the protein sample.

17. The method of claim 14, wherein the SmB polypeptide is bound to a solid support and contacting results in a WDR11/SmB complex bound to the solid support.

18. The method of claim 14, wherein the SmB protein is labeled with a label having an observable characteristic, and detecting any WDR11 protein comprises detecting the observable characteristic.

19. The method of claim 18, wherein the label is selected from the group consisting of a fluorophore, a chromophore, a radioisotope, an enzyme, and a mass label.

20. A kit for testing for the presence of WDR11 protein in a sample, the kit comprising: a SmB polypeptide and one or more of the group consisting of an anti-WDR11 antibody, an anti-SmB antibody, a reagent for sample preparation, and a buffer.


SEQUENCE LISTING (2 sequences)

SEQ ID NO: 1 - protein, 51 aa, Artificial Sequence
  exemplary (imaginary/fictional/no ′real-world′ source) sequence for identity/similarity examples
RTPSDKPVAHVANPQLQWLNRRANALLANGVERDNQLVVEGLYLIYSQVLF

SEQ ID NO: 2 - protein, 52 aa, Artificial Sequence
  exemplary sequence for identity/similarity examples
RAPFKKSWAYLQVAKHKLSWNKDGILHGVRYQDGNLVIQFPGLYFIICQLQF